(19) United States
(12) Patent Application Publication
(10) Pub. No.: US 2024/0206725 A1
(43) Pub. Date: Jun. 27, 2024

(54) OPTOMETRIC TESTING DEVICE AND PROCESS

(71) Applicant: Essilor International, Charenton-Le-Pont (FR)

(72) Inventors: Martha HERNANDEZ-CASTANEDA, Paris (FR); Cécile PETIGNAUD, Villejuif (FR); Denis ROUSSEAU, Gieres (FR); Stéphane PERROT, Saint Maur des Fosses (FR); Gildas MARIN, Paris (FR)

(73) Assignee: Essilor International, Charenton-Le-Pont (FR)

(21) Appl. No.: 18/555,652

(22) PCT Filed: Apr. 26, 2022
(86) PCT No.: PCT/EP2022/061089

§ 371 Date: Oct. 16, 2023

(30) Foreign Application Priority Data
Apr. 28, 2021 (EP) 21305549.4

(51) Int. Cl. A61B 3/08; A61B 3/00; A61B 3/028

(52) U.S. Cl. CPC A61B 3/08; A61B 3/005; A61B 3/0285

(57) ABSTRACT

An optometric testing device comprising a first shutter and a second shutter placed in front of two eyes of a patient and configured to present an activated state in which a shutter blocks the propagation of light and a deactivated state in which the shutter allows the propagation of light, a communication unit configured to communicate with a display system in order to send and/or receive a synchronization signal, and a control unit configured to command each shutter as a function of the synchronization signal so that the first shutter and the second shutter change state with a first frequency and a second frequency, wherein the control unit is configured to command each shutter so that the first frequency is different from the second frequency and/or so that a duty cycle of activation of the first shutter is different from a duty cycle of activation of the second shutter.

### TECHNICAL FIELD OF THE INVENTION

The present invention generally relates to the field of eyeglasses.

More precisely, it relates to an optometry device and process for testing an individual's eyes.

The invention applies to a phoropter comprising such a device and to a pair of eyeglasses comprising such a device.

### BACKGROUND INFORMATION AND PRIOR ART

Known devices and methods for binocular testing of the eyes of a patient are usually dedicated to testing the eyes in far vision conditions. In far vision conditions, the image shown to the patient to test his binocular far vision is usually placed at over 1 meter from the patient's eyes.

However, the refraction features of the eyes in near vision conditions may be different from the refraction features in far vision. This may be due, among other things, to the fact that the accommodation and convergence of the eyes are different in near vision conditions.

Moreover, known methods for testing eyes in near vision are either straight forward (not natural) or with trial frames, not in well controlled conditions or not comfortable for the patient and not easy to control for the practitioner.

### SUMMARY OF THE INVENTION

In this context, the present invention provides a device able to perform vision test in near vision, with different gaze orientations, in binocular vision and optionally in monocular vision, up to a good acuity, and that can be portable.

The device is in fact designed to perform measurements simultaneously on both eyes of the patient. It is also designed to perform these measurements in different situations, that is to say with different postures, these postures preferably corresponding to the posture that an individual adopts in his everyday life (for example when using a phone or a computer, when he reads, when he walks . . . ).

To this end, the invention proposes an optometric testing device to be used together with a mobile display system, comprising:
a first shutter and a second shutter suitable to be respectively placed in front of the two eyes of a patient, each of them being able to present at least two states: an activated state in which the shutter blocks the propagation of light from the display system to the respective eye, and a deactivated state in which the shutter allows the propagation of light from the display system to the respective eye,
a communication unit adapted to communicate with the display system in order to send and/or receive a synchronization signal,
a control unit designed to command each shutter as a function of said synchronization signal so that at least one of the first shutter and the second shutter changes of state respectively with a first frequency at least equal to 60 Hz and a second frequency,
wherein said control unit is programmed to command each shutter as a function of said synchronization signal so that the first frequency is different from the second frequency and/or so that a duty cycle of activation of the first shutter is different from a duty cycle of activation of the second shutter.

Thanks to the invention, the device is able, when two distinct images are alternatively displayed by the display system according to the synchronization signal, to respectively show the two images to the two eyes of the individual, by closing one shutter and opening the other at a frequency that depends on the frequency of change of the images. Thus, the device is designed to perform optometric tests in optimal conditions for binocular vision in near vision.

It also presents the advantage to be used with any kind of mobile display device, for example one belonging to a patient or to an optometrist.

Thus, it can be used to realize the optometric test in the natural, preferred, comfortable or usual posture of the patient. Indeed, the patient may adjust himself the distance between himself and the mobile display, the orientation (angle) of his gaze towards the mobile display in its natural, preferred, comfortable or usual posture. This natural posture condition is very important to obtain the most useful results from the optometric test for day-to-day tasks.

In particular, the device may be designed to perform measurements in binocular vision in order to determine the individual's near vision correction when associated with a phoropter or trial lenses.

The use of such shutters is a simple solution to implement. They can be embedded in a pair of eyeglasses to form a portable device. They can also be embedded in a phoropter suitable to pivot. Consequently, in both embodiments, the device is adapted to perform measurements with different gaze orientations such as downward or straight forward orientation.

The use of different frequencies and/or duty cycles makes it possible to adjust the contrast of the displayed images, the contrast of the two images seen being different. This enables to have a better combination of these images in a 3D image. For example, if the individual has a dominant eye, which prevents him from seeing the two images well fused, by adding a reduced contrast optotype on the image seen by the dominant eye, it is possible to improve the combination of the two images into a 3D image well seen by the patient.

In a more general manner, it is possible to adjust the contrast of the image seen by an eye relative to the contrast of the image seen by the other eye in order to improve the combination of the two images into a 3D image well seen by the patient (considering the patient's subjective perception of this image) or in order to perform special measurements.

It is possible to adjust the contrasts by using the shutters or by using both the shutters and the display system (by adjusting the luminosity of the images or the contrast of the displayed images).

Other preferred features of the invention are the following ones:
said control unit is designed to command each shutter in such a manner that one of said first and second shutters is commanded in deactivated state when the other shutter is commanded in activated state.
said control unit is designed to command each shutter so that the first shutter and the second shutter alternatively change of state.
the device comprises said display system.
said display system includes a display device, and a controller having a driver programmed to command the displaying of a first image and a second image so that the change of state of both shutters is synchronized with the change of displayed image.
said driver is programmed to generate the synchronization signal in accordance with the change of displayed image.
said communication unit is adapted to receive the synchronization signal in the form of a light code, at least one of said first and second of the images comprising this light code.
said display device comprises pixels, the side of each pixel measuring less than 100 microns, and preferably less than 60 microns.
said display device has a minimum vertical dimension of at least 56 mm and a minimum horizontal dimension of at least 56 mm.
said display device presents an afterglow duration equal to or less than 8 ms, preferably less than 6 ms and more preferably less than 3 ms.
said display system is a mobile phone or a tablet.
said communication unit comprises a cable connected to the control unit and adapted to be connected to the display system.
said cable is adapted to be connected to an audio output of said mobile phone.
said communication unit comprises a wireless communication unit suitable to communicate with said display system through radio waves.
said image system displaying images showing a code, said communication unit comprises means for reading said code and means for deducing therefrom said synchronization signal.
each shutter comprises a plate suitable to be commanded between a transparent state and an opaque state.
each shutter comprises a first polarizer and a second polarizer, the first polarizer being situated between the display system and one of the patient's eyes and having a polarizing direction distinct from the one of the first polarizer of the other shutter, the second polarizer being an active polarizing plate situated between the display system and at least one of the patient's eyes.
the second polarizer is adapted to be placed onto the mobile phone screen.
the device includes a phoropter having two optical inputs suitable to be placed in front of the two patient's eyes, and wherein the two shutters respectively belong, at least in part, to the two optical inputs.
in a variant, the device includes a pair of eyeglasses having two lenses, and wherein the two shutters respectively belong, at least in part, to the two lenses.
said shutters are mobile between at least two test configurations: a horizontal vision test configuration where the optical axes extending from the eyes through the shutters extend horizontally and an inclined vision test configuration where said optical axes are inclined downwards.
the optical pathways extending from the display system until the patient's eyes have an optical length of 40 centimeters or less, allowing testing the eyes of the subject in near vision.
the optical pathways extending from the display system until the patient's eyes have an optical length lying from 50 to 70 centimeters, allowing testing the eyes of the subject in intermediate vision.
said display system is programmed to display images having a central optotype and a peripheral part showing at least one element distinct from the optotype.

The invention also relates to an optometric testing process performed by means of an optometric testing device as defined above, comprising steps of:
a) displaying a first image for a first eye of the patient and simultaneously sending a first synchronization signal,
b) when receiving the first synchronization signal, commanding a respective first shutter in an activated state, and the second shutter in a deactivated state,
c) waiting for a time delay expiring, then,
d) displaying a second image, distinct from the first image, for a second eye of the patient and simultaneously sending a second synchronization signal,
e) when receiving the second synchronization signal, commanding the second shutter in an activated state, and the first shutter in a deactivated state,
said steps a) to e) being performed in loop, at a frequency equal or higher than 30 Hz.

### DETAILED DESCRIPTION OF EXAMPLE(S)

The following description with reference to the accompanying drawings, given by way of non-limiting example makes it clear what the invention consists in and how it can be reduced to practice.

In the accompanying drawings:

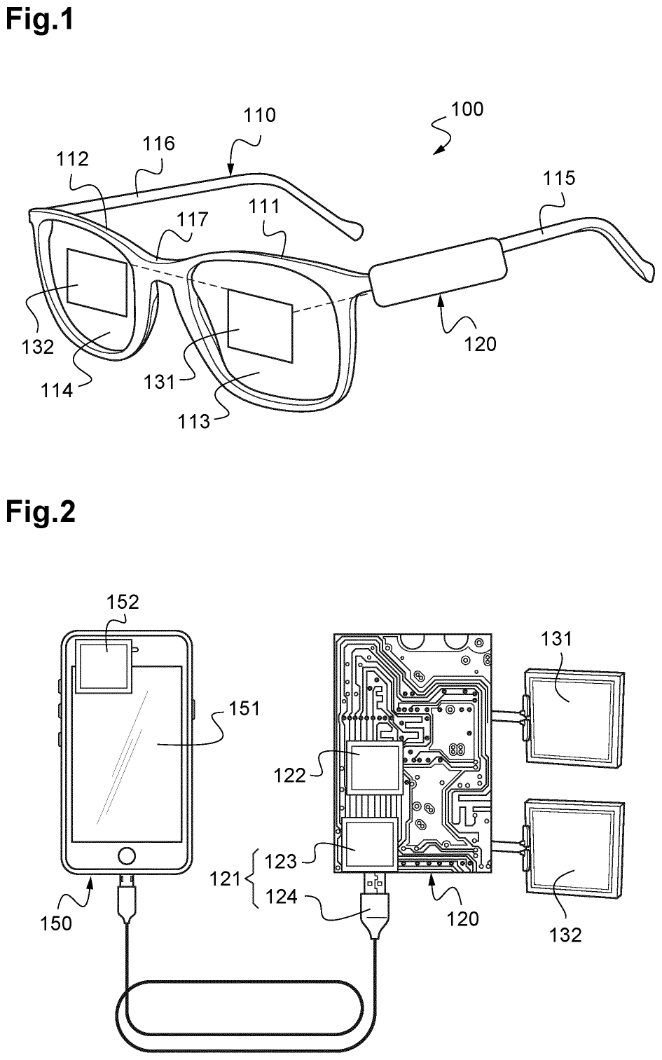

FIG. 1 is a schematic view of a pair of eyeglasses according to a first embodiment of the invention,

FIG. 2 is a schematic view showing two shutters of said pair of eyeglasses and a mobile phone,

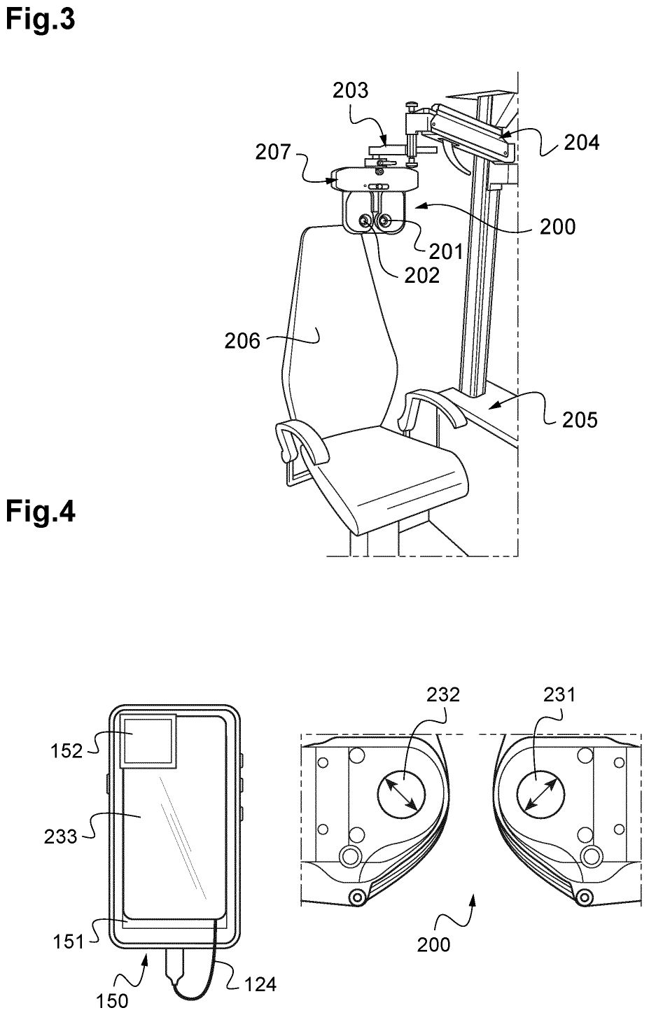

FIG. 3 is a schematic view of a phoropter according to a second embodiment of the invention,

FIG. 4 is a schematic view of the head of the phoropter of FIG. 3 and of a mobile phone,

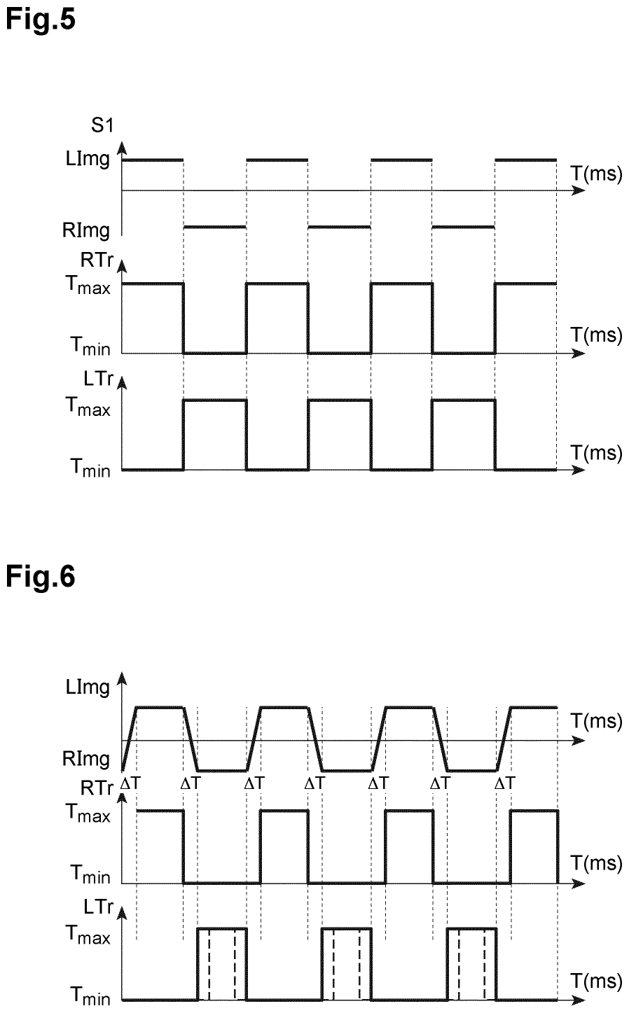

FIGS. 5 to 8 each show three graphs, a first graph representing the alternation of displayed images, a second graph representing the control signal of a first shutter and a third graph representing the control signal of a second shutter,

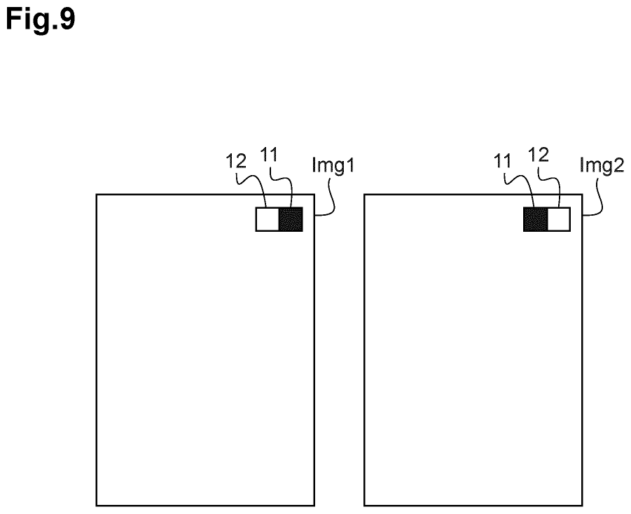

FIG. 9 is a schematic view of two successive images displayed by the mobile phone of FIG. 2.

The invention relates to a device to be used by ophthalmic professionals in order to do optometric measurements (or to do demonstrations) in near vision (and optionally in intermediate vision), in ergonomic conditions (with natural postures that is to say with horizontal gaze and downward gaze), in binocular vision (and optionally in monocular vision), and with a good control of accommodation.

We note that “accommodation” is the process by which the eye of a patient changes optical power to maintain a clear image or focus on an object as its distance varies.

“Horizontal gaze” is a condition in which the direction of the gaze of the patient extends horizontally.

“Downward gaze” is a condition in which the direction of the gaze of the patient does not extend horizontally but is tilted downwards of at least 10 degrees, from his eyes toward the object he looks at.

“Optometric test” may be various possible tests such as measurements of visual acuities, measurements of dissociated phorias, measurements of fusional reserve, measurements of motility errors, optometric tests to do demonstrations or re-education or reinforcement tests to re-educate or re-inforce the accommodation-convergence control of the visual system.

“Monocular vision” is a type of vision in which the patient uses only one of his two eyes to perceive a two-dimensional image of its surroundings.

“Binocular vision” is a type of vision in which the patient uses his two eyes to perceive a single three-dimensional (3D) image of its surroundings.

To perform measurements in Binocular vision, the patient has both eyes opened and un-obstructed, and each eye sees an image different from the other eye.

More precisely, during said binocular measurement, each eye of the patient is provided with a test image. The test images provided to both eyes are configured such that the fusion of the two test images by the brain of the subject may occur. In order to achieve this end, each test image is configured to be accurately aligned with the corresponding eye of the subject.

The optometric testing device according to the invention (named below “device”) is designed to precisely measure binocular vision parameters and optionally monocular vision parameters.

Such parameters comprise the patient's optical prescription. These parameters comprise for instance a spherical power, a prism power, a cylindrical power together with an angle . . . .

Such parameters also comprise the results of the other tests (dissociated phorias, fusional reserve, motility errors . . . ).

The device according to the invention is designed to be used together with a mobile display system, and it comprises means for carrying two different images displayed by this system to the two eyes of the patient.

This device is thus configured for providing a first image to the left eye of the patient (named hereinafter “left image”) and, roughly at the same time, for providing a second image to the right eye of the patient (named “right image”), the right image being preferably different from the left image.

In FIGS. 1 and 2, a first embodiment of this device 100 is shown. In FIGS. 3 and 4, a second embodiment of the device 200 is shown. In both embodiments, the device 100 (resp. 200) comprises a left shutter 131 (resp. 231+233) and a second shutter (resp. 232+233) suitable to be located in front of the two eyes of the patient, each of them being able to present at least two states:
an activated state in which the shutter blocks the propagation of light from the display system to the respective eye, and
a deactivated state in which the shutter allows the propagation of light from the display system to the respective eye.

Thanks to these shutters, when the display system 150 displays a left Image, only the left eye of the patient sees this image (because only the left shutter is in activated state), and vice versa.

The device 100 (resp. 200) also comprises a communication unit adapted to communicate with the display system 150 in order to send or receive a synchronization signal S1, and a control unit programmed to command each shutter as a function of said synchronization signal S1 so that the shutters change of state in a regular pace deduced from the synchronization signal S1.

The curve representing the change of state of the left shutter will be named hereinafter the “left shutter signal”. The curve representing the change of state of the right shutter will be named hereinafter the “right shutter signal”. These signals can be either theoretical or equal to the signals sent to the shutters to make them change of state.

According to the invention, the control unit is programmed to command each shutter as a function of said synchronization signal S1 so that:
the frequency of each left and right shutter signal is at least equal to 60 Hz, and
the frequencies and/or the duty cycle of these signals are different.

In a preferred embodiment, both frequencies are identical, and the duty cycles are different.

The duty cycle of a signal is defined as the fraction of one period in which the shutter is not in the deactivated state.

The control unit is preferably programmed to command each shutter so that the first shutter and the second shutter alternatively change of state.

In other words, each shutter is alternatively commanded between the activated state and the deactivated state, on a regular pace defined by a frequency f1 (for the left shutter) or f2 (for the right shutter).

We note that preferably, one of the ratios between the frequencies f1 and f2 (f1/f2 or f2/f1) is an integer.

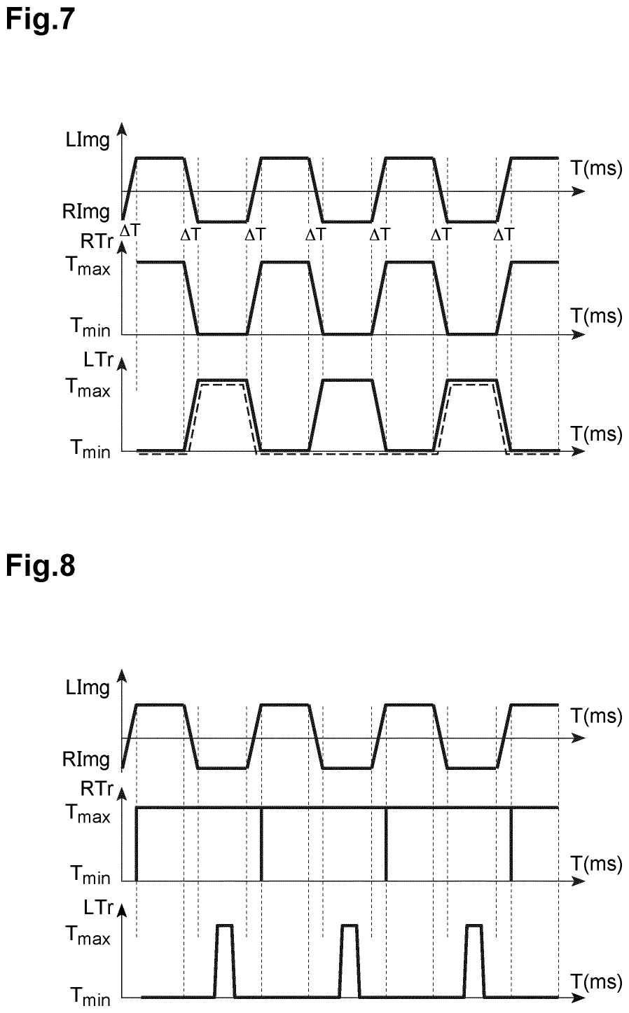

The shutter signals have preferably both a square shape (FIGS. 5, 6 and 8) or a trapezoid shape (FIG. 7).

As shown in the represented figures, the device 100 (resp. 200) according to the invention can have the shape of an eyeglasses (first embodiment) or of a phoropter (second embodiment) or any other shape.

The shutters can be formed by screens (first embodiment) or can be based on a polarization technology (second embodiment) or can present any other form.

The measurements are performed by means of this device 100 (resp. 200), together with a display system that can belong or not to the device. In other words, the display system can be dedicated to such measurements or not.

The display system comprises a display device. This display device is preferably a screen but, in a variant, it may be a projector, for instance a pico-projector (that may be embedded in an eyeglasses frame), a LCOS projector, a laser projector coupled to MEMS mirrors . . . .

In the preferred embodiment, the display device can be a LCD screen, a OLED screen, or other. This screen will be described in more detail hereinafter.

In the shown embodiments, this display system is a mobile phone 150 that comprises a touch screen 151 and that is programmed to perform the process according to the invention.

At this step, we can describe in more details the first embodiment of the device 100, referring to FIGS. 1 and 2.

As shown in FIG. 1, the device 100 comprises a pair of eyeglasses 110, a print circuit board 120 embedded in this pair of eyeglasses, and two shutters 131, 132.

The pair of eyeglasses includes two ophthalmic lenses 113, 114, two temples 115, 116 and a nose pad 117. In the shown example, it also includes two rims 111, 112, connected by the nose pad 117, in which the lenses 113, 114 are respectively mounted. The temples are respectively articulated on the two rims.

The two shutters 131, 132, are respectively located in the two lenses 113, 114. To this end, they are here glued onto the lenses.

In a variant, the two shutters may be clipped on the frame.

In another variant, the two shutters may be connected to each other by a nose pad, to form a kind of spectacles without temples.

Each shutter is a liquid crystal device comprising one or several pixels that can switch between the activated state (a transparent state) and the deactivated state (a black state). If a shutter comprises more than one pixel, all these pixels are all driven in the same state, at any time. In other words, when a shutter is in the activated state, all its pixels are transparent and when the shutter is in the deactivated state, all its pixels are black.

The used technology is chosen to enable a rapid change of state (in less that 1 ms*).

Here, the shutters 131, 132 are sold by Swedish LC-Tec company under the code “X-FOS(G2)”.

The print circuit board 120 comprises several circuits. It comprises a communication unit 121 adapted to communicate with the mobile phone 150 in order to receive the synchronization signal S1, and the control unit 122 programmed to command each shutter as a function of said signal.

The communication unit 121 comprises a circuit 123 that receives from the mobile phone 150 the synchronization signal S1 (here y means of a cable 124), and sends this signal to the control unit 122.

The control unit 122 comprises a processing unit, such as a CPU, a programmable logic device (DSP, FGPA . . . ) or a controller, or any combination thereof. It also comprises a memory and various input and output interfaces.

Thanks to its input interfaces, the control unit 122 is suitable for receiving the synchronization signal S1.

Thanks to its output interfaces, the control unit 122 is suitable for controlling the shutters 131, 132.

Thanks to its memory, the control unit 122 stores a computer application, consisting of computer programs comprising instructions, the execution of which by the processor enables the control unit 122 to implement the process described below for selectively conveying the left and right images to the left and right patient's eyes. In the variant where the control unit 122 comprises a programmable logic device, this device comprises logic gates designed to perform the process described below.

In a preferred embodiment, this control unit 122 is programmed so that:
when the image for the right eye of the patient is displayed, the right shutter 132 is driven in the activated state and the other shutter is driven in the deactivated state, and
when the image for the left eye of the patient is displayed, the left shutter 131 is driven in the activated state and the right shutter 132 is driven in the deactivated state.

Because the two images are produced in the same single display zone of the single screen 151 in an alternating manner, that is to say one after the other, using all the display zone of this screen, the shutters enable to convey alternatively these images to the two eyes at a high frequency.

Now, we can describe in more details the second embodiment of the device 200, referring to FIGS. 3 and 4.

In this embodiment, the device 200 is a phoropter head adapted for determining refractive properties or refractive correction need for the eyes of the patient who is a wearer of corrective eyeglasses or contact lenses whose correction needs are to be assessed.

In a classic way, the phoropter head is mounted on a holder 203 which is further linked to a hinged arm 204. The hinged arm 204 is further attached to a stationary portion of the phoropter 205. When assessing the correction needs of the patient, said patient is seated in a seat 206, and eyepieces 201, 202 of the phoropter head are placed in front of the patient's eyes. The patient's correction needs are evaluated based on the aptitude of the patient to identify the characters displayed on the mobile phone screen 151 when he looks through optical systems arranged behind the eyepieces 201, 202.

Each of the eyepieces 201, 202 of the phoropter head 200 delimits an optical input through which the patient can look at the screen 151 of the mobile phone 150 by one of his eyes.

Each optical system arranged in an eyepiece 201, 202 comprises a lens with variable power. It comprises here a deformable liquid lens having an adjustable shape. Alternatively, or in addition, the optical system may comprise an ensemble of non-deformable lenses having different optical powers, and a mechanical system that enables to select some of these lenses to group them to form the set of lenses through which the subject can look.

The distance between the eyepieces 201, 202 can be adjusted according to the distance between the two eyes of the patient.

Here, the phoropter head is mounted on the holder 203 via a pivot linkage allowing it to pivot about a horizontal pivot axis orthogonal to the optical axes of the eyepieces 201, 202. The linkage between the phoropter head and the holder 203 allows the phoropter head to pivot between a horizontal position in which the patient's gaze is horizontal and a tilted position in which the patient's gaze is tilted downwards.

In this second embodiment, each shutter comprises at least two polarizers: a first polarizer 231, 232 and a second polarizer 233.

As shown in FIG. 4, the first polarizer 231 of the left shutter belongs to the left eyepiece 201 of the phoropter head, and the first polarizer 232 of the right shutter belongs to the right eyepiece. More specifically, each optical system arranged in an eyepiece 201, 202 comprises a transparent passive polarizing plate. The polarizing direction of the two first polarizers 231, 232 are distinct and are preferably orthogonal to each other.

In the embodiment shown in FIG. 4, the second polarizer 233 is an active polarizing plate that is designed in order to be placed on the screen 151 of the mobile phone 150.

The second polarizer 233 is active in the sense that its polarizing direction can be controlled, here by the mobile phone 150. This polarizer 233 has a first state in which a linear polarization is unchanged and a second state in which the linear polarization is tilted by 90°.

In a preferred variant, the second polarizer 233 is a twisted nematic plate, the front polarizer of which is removed.

This second polarizer is common to the two shutters but in a variant, each shutter could comprise its own active polarizing plate, located for instance on the eyepieces 201, 202 of the phoropter head.

The processing unit of the mobile phone 150 forms the control unit 152 that drives the polarizing direction of the second polarizer 233.

To this end this control unit 152 memorizes a driver programmed to command alternatively the displaying of a first image and a second image and to generate and send the synchronization signal S1 to the second polarizer 233 so that the change of state of the shutters (each formed of two polarizers) is synchronized with the change of displayed image.

More specifically, the controller 152 is programmed so that:
when the image for the right eye of the patient is displayed, the polarizing direction of the second polarizer 233 is approximately parallel to the polarizing direction of the first polarizer 232 of the right shutter (in this state, the polarizing direction of the second polarizer 233 is approximately orthogonal to the polarizing direction of the first polarizer 232 of the left shutter), and
when the image for the left eye of the patient is displayed, the polarizing direction of the second polarizer 233 is approximately parallel to the polarizing direction of the first polarizer 232 of the left shutter.

We can note that, because the first polarizers 231, 232 of the shutters are located on the phoropter head 200, they are mobile between two test configurations: a horizontal vision test configuration where the optical axes extending from the eyes through the shutters are horizontal and an inclined vision test configuration where said optical axes are inclined downwards.

In both represented embodiments, the screen 151 that is used to display the optotype belongs to a mobile phone 150. As explained above, in a variant, this screen could be dedicated to the measurement of the binocular and monocular vision parameters.

This screen 151 has the following specifications:
dimensions that are suitable to give a field of view of 8° in horizontal (and in vertical if possible),
pixel dimensions that allow the measurement of a visual acuity of at least 8/10, preferably 10/10, more preferably 15/10 and ideally 20/10,
a frequency of at least 60 Hz and preferably 90 Hz or 120 Hz or 240 Hz,
an image displaying having little afterglow, the duration of this afterglow preferably being lower than ⅕ of the duration of the displaying of each image, and being more preferably lower than 1/10 of this duration.

Here, to fulfill these specifications, the screen has the following characteristics:
dimensions of at least of 56 mm in horizontal and vertical,
pixel dimensions lower than 50 microns,
a screen frequency of at least 60 Hz, and
an afterglow duration lower than 3 ms. Preferably, the duration of the afterglow of each displayed image is below 1.66 ms.

In both represented embodiments, the synchronization between the shutters and the mobile phone 150 can be performed in various ways.

As shown in FIGS. 2 and 4, the communication unit comprises a cable 124 connected between the shutters and the mobile phone 150. More specifically, in the first embodiment, the cable is connected, on the one hand, to the mobile phone 150, and, on the other hand, to the printed circuit board 120. In the second embodiment, it is connected to the second polarizer 233.

This cable is for instance an USB cable connected to the main socket of the mobile phone 150. The use of such a cable is preferred because this solution has the advantage to be almost instantaneous.

In a variant of the first embodiment, the cable is connected to an audio output of the mobile phone. In this variant, the control unit 152 of the mobile phone 150 is programmed to generate a synchronization signal S1 that is an audio signal. In this variant, the control unit 122 of the printed circuit board 120 has to transform the received audio signal into an input signal for the shutters 131, 132.

In a second variant, the communication unit does not comprise any cable but includes a wireless communication unit suitable to communicate with the mobile phone 150 through radio waves. For instance, the shutters 131, 132 (FIG. 2) or the second polarizer 133 (FIG. 4) can be synchronized with the mobile phone 150 by Bluetooth, Wifi or Infrared light.

In a third variant applicable to the first embodiment, all the images displayed on the mobile phone screen 151 show a code, this code being different for the left images and for the right images. The communication unit comprises means for reading the code and means for deducing therefrom the synchronization signal S1. Different solutions are conceivable to execute this variant. For instance, the mobile phone 150 can integrate a white square in a corner of the right images, and a black square on all the left images. The pair of eyeglasses can comprise a photoreceptor or a camera situated in front of the corner of the displayed images. The photoreceptor or the camera, when the images for the left eye are displayed, receives a signal completely different from the signal received when the images for the right eye are displayed. This synchronization signal is sent to the control unit 122 to transform this signal into an input signal for the shutters. This third variant will be described in more details hereinafter.

In both embodiments, the device 100, 200 can perform a testing process comprising five main steps.

The first step consists in displaying on the mobile phone screen 151 a left image for the left eye of the patient and simultaneously generating a first pulse in the synchronization signal S1.

The second step consists, when this first pulse is received and detected, in driving the left shutter in an activated state, and the right shutter in a deactivated state.

Then, during a third step, the expiration of a time delay is waited for.

After, during a fourth step, a right image for the right eye of the patient is displayed and a second pulse of the synchronization signal S1 is simultaneously generated.

Finally, during a fifth step, when this second pulse is received and detected, the right shutter is driven in an activated state, and the left shutter in a deactivated state.

These five steps are performed in loop, at a frequency equal or higher than 30 Hz.

The displayed images preferably have a central optotype and a peripheral part showing at least one element distinct from the optotype.

The duration of activation of the shutters depends on the synchronization signal S1, which depends on the kind of image displayed (left or right).

In FIG. 5, the first upper graph represents the synchronization signal S1, that is to say the variation of image displayed on the mobile phone screen 151 as a function of the time T. When this signal is in high state, a left image LImg is displayed and when it is in low state, a right image RImg is displayed.

The right shutter signal RTr is shown in the second graph of FIG. 5 and the left shutter signal LTr is shown in the third lower graph. These graphs represent a theoretical example of variation of the shutter signals, and more precisely a situation that does not belong to the scope of the invention but that is represented to illustrate the principle of the invention.

These signals have square shapes. They vary between a high value Tmax and a low value Tmin. The high value Tmax is the maximum transmittance of the shutter and the low value Tmin is the minimum transmittance of the shutter.

In this Figure, the frequency f1 of the left shutter signal RTr is equal to 60 Hz and the frequency f2 of the right shutter signal RTr is equal to 60 Hz. These signals are in opposition of phase.

In this example, the duration of displaying of each image is of 8.3 ms.

When an image for the right eye is displayed, the right shutter is in the activated state and the other shutter is in the deactivated state. When an image for the left eye is displayed, the left shutter is in the activated state and the other shutter is in the deactivated state.

Consequently, each eye sees only the corresponding image. This enables to display two images in alternance that can fuse to form a 3D image seen by the patient.

But this simplified example does not take into account the fact that the images displayed on the screen 151 cannot always be instantaneously displayed.

The displaying and the erasing (also called afterglow) of the images indeed take time. Because of this, the risk is that the image for the left eye is seen by the other eye, and vice versa.

Consequently, there is a need for taking into account the characteristics of the screen to drive the shutters.

The main characteristic is the duration needed by the screen to erase a previous image and to entirely display a new image.

In FIG. 6, the first graph shows the transition between the displaying of two successive images, the duration of which is referenced ΔT.

In this FIG. 6, the two other graphs, that represent in solid lines the shutters signals, show that the shutters are both driven in the deactivated state during the duration ΔT of this transition.

Consequently, thanks to this strategy, the left eye of the patient can only see the image for the left eye, and vice versa.

FIG. 7 shows a variant that also takes into account the duration needed by the screen to erase a previous image and display a new image entirely.

In FIG. 7, the first graph is identical to the one of FIG. 6.

The two other graphs, that represent in solid line the shutters signals, show that the shutters are controlled to gradually shift from one state to the other state.

More specifically, the control unit is programmed so that:
when the image RImg for the right eye of the patient is entirely displayed, the right shutter is in activated state and the left shutter is in deactivated state,
when the image LImg for the left eye of the patient is entirely displayed, the left shutter is in activated state and the right shutter is in deactivated state,
during the duration ΔT of a transition from the right image to the left image, the right shutter progressively shifts from the activated state to the deactivated state and the left shutter progressively shifts from the deactivated state to the activated state, and
during the duration ΔT of a transition from the left image to the right image, the right shutter progressively shifts from the deactivated state to the activated state and the left shutter progressively shifts from the activated state to the deactivated state.

In the first embodiment, to progressively shift from one state to another, the pixels of the shutter 131, 132 are all commanded to get increasingly darker or clear.

In the second embodiment, to progressively shift from one state to another, the polarizing direction of the second polarizer 233 increasingly varies from one direction to another.

The advantage, compared to the example of FIG. 6, is that the global transmittance of the system is higher, and there is no moment when the patient does not see anything. In other words, there is no moment when all the shutters are closed, which could be uncomfortable for the eyes of the patient.

The examples of FIGS. 6 and 7 shown in solid line do not belong to the scope of the invention but are represented for understanding the invention.

The core of the invention is to adjust the light received by one eye relative to the other eye in binocular vision, to improve the combination of the left and right images by the patient's brain in order to generate a 3D image.

A first advantage is to take into account the fact that one of the eyes of the patient is a dominant eye and that the patient mainly uses this dominant eye to see the displayed images, making him felt some difficulties to see a 3D image.

In this case, the solution is to change the timing of one of the shutters. More specifically, it is possible to change the frequency or the duty cycle of the shutter signal corresponding to the dominant eye, to reduce the luminance of the image sent to this eye relative to the luminance of the image sent to the other eye.

A second advantage is to take into account that the duration of afterglow is different when passing from a dark image to a clear image than when passing from a clear image to a dark image.

Indeed, when passing from a white image to a black image, the new black image appears with a very little afterglow. But, when passing from a black image to a white image, there is an afterglow that generates a grey image.

In this case, the solution is to change the timing of one of the shutters, depending on the displayed images.

In other words, to obtain a different contrast for the two patient's eyes, the activation duration of the left shutter is different than the activation duration of the right shutter.

To this end, each shutter is commanded as a function of the synchronization signal S1 so that the frequency and/or the duty cycle of the right shutter signal is different from the frequency and/or the duty cycle of the left shutter signal.

A first example is represented in dotted line in FIG. 6. In this Figure, the duty cycle of the left shutter signal LTr is reduced relative to the one of the right shutter signal RTr.

A second example is represented in dotted line in FIG. 7. In this figure, the frequency and the duty cycle of the left shutter signal are reduced relative to the ones of the right shutter signal.

A third example is represented in solid line in FIG. 8. In this figure, the duty cycle of the right shutter signal is increased to almost 100% (it is preferably strictly lower to 100%) when the duty cycle of the left shutter signal is much lower.

When the duty cycle of the right shutter signal is equal to 100%, the right shutter remains continuously activated. In this situation, the frequency of this signal is considered equal to 0 Hz.

In a variant of this FIG. 8, we can consider that the images for the left eye and for the right eye are the same. Consequently, there is no afterglow and the duration ΔT is null. In other words, the same image is continuously displayed on the screen.

In this variant, it is possible to activate one shutter for a duration that is different than for the other shutter, as shown by the shutter signals shown on the second and third graphs of FIG. 8.

For instance, the duty cycles for the shutter signals are almost 100% and 5%. In other words, because the frequency is of 60 Hz (the corresponding period is of 16.6 ms), the left shutter can be activated during 1 ms by period when the other shutter is activated during 16 ms by period. Consequently, the patient does not see the same image with his two eyes.

At this step of the description, we can detail how this device and process can be used to perform some interesting measurements.

The measurements are preferably performed in near vision, that is to say when the optical pathways extending from the mobile phone screen 151 until the patient's eyes have an optical length of 40 centimeters or less. To this end, the patient has to maintain his mobile phone 150 in front of his eyes, at a constant distance of 40 centimeters or less.

But the measurements can also be performed in intermediate vision, that is to say when the optical pathways extending from the mobile phone screen 151 until the patient's eyes have an optical length of 50 to 70 centimeters. To this end, the patient has to maintain his mobile phone 150 in front of his eyes, out at arms' length.

In a variant, the mobile phone can be placed on a mechanical support.

The measurements are performed in two test configurations, a horizontal vision test configuration where the optical axes going from the eyes through the shutters extend horizontally and an inclined vision test configuration where said optical axes are inclined downwards (for instance at 30 degrees).

A first possibility with the device (100 or 200) is to display two different images on each eye in order to create retinal disparities that will make objects seen at different depth locations from the screen, thus allowing to evaluate the stereovision of the patient. Since the eyes are always accommodating at the screen distance but converging on the virtual object that may be at a different distance, the system allows to test the capacities of the accommodation-convergence visual system of the patient. Thus, it is possible to perform measurements or re-education exercises.

Another possibility with the device is to dynamically measure the posture of the head and the distance at which the patient fixes the virtual object, for instance by means of motion capture and eye-tracking systems. Thanks to the measured data, it is possible to obtain different visual parameters associated to specific tasks or activities of daily life (working on a computer, reading information on a smartphone, reading a book, writing a letter . . . ).

Another possibility is to perform measurements of monocular visual acuities by displaying optotypes in only the images for one eye. Then it is possible to perform measurements of binocular visual acuities by displaying optotypes at the same location for both eyes, or of stereo acuities by displaying optotypes at different location for each eye, the difference providing a depth perception from the screen (the stereo acuity is the minimum depth perceived).

In this case, it may be interesting to change the luminosity or the contrast for one of the eyes, for example by using one polarizer at 45° for one eye and another at 90° for the other eye.

Another possibility is to perform measurements of dissociated phorias by associating the device with an eye tracker, by switching the optotype display from one eye to the other and by measuring a deviation.

Another possibility is to perform measurements by displaying optotypes in which the difference in location for each eye is increasing until the person sees blurred, then doubled (break point) images and then decreasing until single again (recovery point) images are seen.

It is also possible to re-educate or strengthen the accommodation-convergence control of the patient's visual system by moving slowly the virtual distance of the object or optotype displayed alternatively from far to near and near to far, the accommodative plan still remaining on the screen.

It is also possible to measure motility errors (for instance by means of the Hess-Weiss test). For example, ocular motility can be evaluated in monocular and binocular vision. To this end, a fixation point successively appears on the screen in different positions (primary, secondary and tertiary positions) in order to slow down a motor problem. In order to complete the diagnosis, the Bielschowsky maneuver can be performed (the head leans over each of the shoulders) and the test is repeated making the fixation point appear in different positions on the screen.

The visual field can also be controlled by making a fixation point appear on the screen at randomized different positions.

In the second embodiment (with a phoropter), it is possible to simulate the optical effect of a proposed correction that may be different on each eye.

It is also possible to simulate residual blur and distortion on a virtual scene or on the reality (by using a camera) with methods described in EP2020905 and EP2230988.

It is possible to simulate the impact of a progressive lens.

It is possible to simulate the difference in magnification of lenses with the difference in the eye due to anisometropia or different base curve, the impact of various pairing rules enabling to choose the base curve of each eye.

It is possible to simulate the impact of different blur levels on each eye and compare the impact on the dominant and non-dominant eye, to demonstrate the benefit of a binocularly optimized lens or of a differentiated contact lens correction (for example with one eye for near vision and one eye for far vision).

In the third variant described above and shown in FIG. 9, all the images displayed on the mobile phone screen 151 show a light code (a pattern), this code being different for the left images and for the right images. We can now describe this variant in more details.

In this variant, the screen of the smartphone displays two distinct left and right images img1, Img2 at a high frequency (typically at 120 Hz).

On each image, a specific pattern is displayed in one corner. The place where the pattern is displayed may depend on the method used for reading the images. The shape of the pattern can depend on its place (if the code is displayed on the upper edge of the image, a horizontally elongated pattern is preferred, but if the code is displayed on the right edge of the image, a vertically elongated pattern is preferred).

To distinguish the images, the left images could include only a white square and the right images could include only a black square. The images may be colored in respectively white/black, red/green, red/blue or any other combinations of colors.

But in a preferred embodiment, a double-square with a black square 11 and a white square 12 is used on each image. In this variant, the positions of the black and white squares are reversed for the left and right images Img1, Img2. This embodiment is more accurate, for instance if the displayed image is dark so that the black square cannot be seen by a high-speed camera or if the image is bright. Thanks to these two colors, the code can be read easier, and the communication unit can deduce therefrom the synchronization signal S1 without any error.

The device 100 can comprise a camera for reading the code. In a variant, photocells or other light detectors can be placed in front of the squares, to detect the specific pattern. Each time the detector sees a new pattern, the new image is detected, and the shutters can be activated or deactivated depending on the image.

In other examples, codes of other shapes could be used. In a similar way, the screen could have different light emission modes that cannot be detected by a human eye but that can be detected by the device 100 to distinguish the left image from the right image.

This third variant is particularly interesting for the following reason.

Standard screens can have a frequency that is slightly distinct of the exact frequency that is requested by the software and the operating system: sometimes the display is slower than requested, or do not start exactly when the synchronization signal is issued from the device (for example from the USB output). To be sure to exactly synchronize the shutters with the displayed images, it is important to activate and deactivate the according shutters at the exact time when the new image is displayed. In this third variant, because the synchronization signal S1 depends on the images (and not on any other electrical or audio signal), we can be sure that the synchronization is accurate.

We can note that, to improve the method and to be sure to obtain the best synchronization signal S1, it is possible to build this signal on the basis by combining: firstly, the code shown on the images and; secondly, an electrical or audio signal receive through another canal (for instance by the USB output). This mixed solution increases the accuracy of the system, and avoid great error when one signal is missing.

The present invention is in no way limited to the embodiment described and shown.

In particular, the difference of contrasts between the images seen by the patient's eyes can also be increased by controlling the screen accordingly.

Indeed, when the screen is of the LCD type, it comprises a liquid crystal panel and a backlight made of LEDs. In this variant, these LEDs can be driven synchronously with the liquid crystal panel and the shutters.

For example, first, it is possible to light on the LEDs only when the image displayed on the liquid crystal panel is stable (that is to say when the image is fully displayed). Moreover, it is possible to increase the light intensity of the LEDs when the image for the right eye is displayed by the liquid crystal panel and to reduce it when the image for the left eye is displayed, or vice versa. By doing this synchronously with the shutter control, the image for one eye can have a different contrast as the image for the other eye.

When the screen is of the OLED type, there is no backlight and the panel emits the light. Here again, it is possible to increase the light intensity of the panel when the image for the right eye is displayed and to reduce it when the image for the left eye is displayed, or vice versa.

It should be noted that these variants are not preferred because they require controlling the screen in a particular way and the shutters, whereas it is preferable to control only the shutters to adjust the contrast between the images seen by the patient.

In the second embodiment, the polarizing directions of the two first polarizers 231, 232 are linear and orthogonal to each other, and the second polarizer 233 is active in the sense that its polarizing direction can be controlled. In a variant, the polarizing directions of the two first polarizers 231, 232 can be circular and oriented in opposite directions, and the second polarizer 233 can be active in the sense that its circular polarizing direction can be controlled.

1. An optometric testing device comprising:
a first shutter and a second shutter configured to be placed in front of two eyes of a patient, each of the first shutter and the second shutter being configured to present at least two states, the at least two states including: an activated state in which a shutter blocks propagation of light from a display system to an eye, and a deactivated state in which the shutter allows the propagation of light from the display system to the eye,
communication circuitry configured to communicate with the display system in order to send and/or receive a synchronization signal, and
control circuitry configured to command each shutter as a function of the synchronization signal so that the first shutter and the second shutter change state respectively with a first frequency at least equal to 60 Hz and a second frequency,
wherein the control circuitry is configured to command each shutter as a function of the synchronization signal so that the first frequency is different from the second frequency and/or so that a duty cycle of activation of the first shutter is different from a duty cycle of activation of the second shutter.

2. The optometric testing device according to claim 1, wherein is the control circuitry is further configured to command each shutter in such a manner that one of the first and the second shutters is commanded in the deactivated state when the other shutter is commanded in the activated state.

3. The optometric testing device according to claim 2, wherein is the control circuitry is further configured to command each shutter so that the first shutter and the second shutter alternately change state.

4. The optometric testing device according to claim 1, wherein the display system includes:
a display device, and
a controller having a driver programmed to command displaying of a first image and a second image so that a change of state of both the first shutter and the second shutter is synchronized with a change of a displayed image.

5. The optometric testing device according to claim 4, wherein the driver is programmed to generate the synchronization signal in accordance with the change of the displayed image.

6. The optometric testing device according to claim 4, wherein the communication circuitry is further configured to receive the synchronization signal in the form of a light code, at least one of the first and the second image comprising the light code.

7. The optometric testing device according to claim 1, wherein the display system is a mobile phone or a tablet.

8. The optometric testing device according to claim 1, wherein the communication circuitry comprises a cable connected to the control circuitry and configured to be connected to the display system.

9. The optometric testing device according to claim 1, wherein the communication circuitry is configured to communicate with the display system through radio waves.

10. The optometric testing device according to claim 1, wherein each shutter comprises a plate configured to be commanded between a transparent state and an opaque state.

11. The optometric testing device according to claim 1, wherein each shutter comprises a first polarizer and a second polarizer, the first polarizer of one of the shutters being situated between the display system and one of the two eyes of the patient and having a polarizing direction distinct from a polarizing direction of the first polarizer of the other shutter, the second polarizer being an active polarizing plate situated between the display system and at least one of the two eyes of the patient.

12. The optometric testing device according to claim 11, wherein the second polarizer is configured to be placed onto a screen of a mobile phone.

13. The optometric testing device according to claim 1, further comprising a phoropter having two optical inputs configured to be placed in front of the two eyes, and wherein each shutter belongs, at least in part, to one of the two optical inputs.

14. The optometric testing device according to claim 1, further comprising a pair of eyeglasses having two lenses, and wherein each shutter belongs, at least in part, to one of the two lenses.

15. An optometric testing process performed by an optometric testing device having a first shutter and a second shutter configured to be placed in front of two eyes of a patient, each of the first shutter and the second shutter being configured to present at least two states, the at least two states including: an activated state in which a shutter blocks propagation of light from a display system to an eye, and a deactivated state in which the shutter allows the propagation of light from the display system to the eye, communication circuitry configured to communicate with the display system in order to send and/or receive a synchronization signal, and control circuitry configured to command each shutter as a function of the synchronization signal so that the first shutter and the second shutter change state respectively with a first frequency at least equal to 60 Hz and a second frequency, wherein the control circuitry is configured to command each shutter as a function of the synchronization signal so that the first frequency is different from the second frequency and/or so that a duty cycle of activation of the first shutter is different from a duty cycle of activation of the second shutter, comprising:
a) displaying a first image for a first eye of the patient and simultaneously sending a first synchronization signal,
b) commanding, in response to receiving the first synchronization signal, the first shutter to be in the activated state, and the second shutter to be in the deactivated state,
c) waiting for a time delay to expire,
d) displaying a second image, distinct from the first image, for a second eye of the patient and simultaneously sending a second synchronization signal, and
e) commanding, in response to receiving the second synchronization signal, the second shutter to be in the activated state and the first shutter to be in the deactivated state,
wherein a) to e) are repeated at a frequency equal to or higher than 30 Hz.

16. The optometric testing device according to claim 2, wherein the display system includes:
a display device, and
a controller having a driver programmed to command displaying of a first image and a second image so that a change of state of both the first shutter and the second shutter is synchronized with a change of a displayed image.

17. The optometric testing device according to claim 2, wherein each shutter comprises a plate configured to be commanded between a transparent state and an opaque state.

18. The optometric testing device according to claim 2, wherein each shutter comprises a first polarizer and a second polarizer, the first polarizer of one of the shutters being situated between the display system and one of the two eyes of the patient and having a polarizing direction distinct from a polarizing direction of the first polarizer of the other shutter, the second polarizer being an active polarizing plate situated between the display system and at least one of the two eyes of the patient.

19. The optometric testing device according to claim 18, wherein the second polarizer is configured to be placed onto a screen of a mobile phone.

20. The optometric testing device according to claim 2, further comprising a phoropter having two optical inputs configured to be placed in front of the two eyes, and wherein each shutter belongs, at least in part, to one of the two optical inputs.
